(19) United States
(12) Patent Application Publication
(10) Pub. No.: US 2026/0294840 A1
(43) Pub. Date: Oct. 1, 2026

(54) COMPOSITIONS AND METHODS USING THYMOL AND/OR CARVACROL FOR INDUCTION OF AUTOPHAGY

(71) Applicant: SOCIETE DES PRODUITS NESTLE S.A., Vevey (CH)

(72) Inventors: JEROME FEIGE, Crissier (CH); PHILIPP GUT, Geneve (CH); GABRIELE CIVILETTO, Lausanne (CH); FEDERICO SIZZANO, Ferreyres (CH)

(21) Appl. No.: 19/706,429

(22) Filed: Jun. 12, 2026

Related U.S. Application Data
(63) Continuation of application No. 17/596,779, filed on Jun. 19, 2020.

(30) Foreign Application Priority Data
Jun. 20, 2019 (EP) 19181530.7

(51) Int. Cl. A61K 31/05; A61K 31/132; A61K 31/155; A61K 31/20; A61K 31/37; A61K 31/436; A61K 31/496; A61K 31/522; A61P 3/04

(52) U.S. Cl. CPC A61K 31/05; A61K 31/132; A61K 31/155; A61K 31/20; A61K 31/37; A61K 31/436; A61K 31/496; A61K 31/522; A61P 3/04

(57) ABSTRACT

Compositions and methods can use a combination of thymol and/or carvacrol, optionally with high protein for induction of autophagy in an individual in need thereof. Preferably, a formulation containing a combination of thymol and/or carvacrol, optionally with protein is administered to the individual in an amount effective to induce autophagy, for example in muscle. The formulation can concomitantly promote protein synthesis and removal of damaged cellular materials.

### CROSS REFERENCE TO RELATED APPLICATIONS

The present application is a continuation of U.S. patent application Ser. No. 17/596,779 filed Dec. 17, 2021, which is a National Stage of International Application No. PCT/EP 2020/067250 filed Jun. 19, 2020, which claims priority to European Patent Application No. 19181530.7 filed Jun. 20, 2019, the entire contents of which are incorporated herein by reference.

### BACKGROUND

The present disclosure generally relates to compositions and methods which use a combination of thymol and/or carvacrol for induction of autophagy. More specifically, the present disclosure relates to administering a formulation comprising thymol and/or carvacrol, alone or in combination with high protein, in an amount effective to induce autophagy, for example in muscle. The formulation can concomitantly promote protein synthesis and removal of damaged cellular materials. The recipient of administration can be an individual or a critically ill patient, for example a patient in the Intensive Care Unit (ICU), an ageing patient, for example an elderly individual or a patient with sarcopenia or frailty ; or an individual with chronic kidney disease (e.g., with a loss of amino acids from dialysis) and/or acute kidney injury, or liver disease.

Due to major advances in intensive care medicine, critically ill patients often survive acute conditions that were previously lethal. Nevertheless, mortality remains high in these patients who survive this initial phase and enter a chronic phase of critical illness. Mortality is often from non-resolving multiple organ failure, acute kidney injury and failure, critical illness myopathy, or less severe forms of muscle weakness. Treatments have been introduced to improve muscle myopathy and weakness, such as hyperalimentation, growth hormone, or androgens, but have failed because these interventions unexpectedly increased the risk of organ failure and death. Moreover, the nutritional support to trauma and surgery patients may actually have detrimental effects.

Effective measures to provide critically ill patients with appropriate treatments and adequate nutrition remain lacking.

### SUMMARY

The degradation of cytoplasmic proteins is mediated by a cellular process referred to as macroautophagy, also referred to simply as autophagy. Autophagy processes are also involved in the inflammatory response and facilitate immune system destruction of bacteria. Autophagy constitutes the major lysosomal degradation pathway recycling damaged and potentially harmful cellular material such as damaged mitochondria. Notably, autophagy counteracts cell death and prolongs life span in various ageing models. The inventors surprisingly found that thymol and/or carvacrol induces muscle autophagy. Moreover, thymol and/or carvacrol synergistically induces muscle autophagy in combination with a high protein isocaloric diet.

Accordingly, in a general embodiment, the present disclosure provides a composition comprising thymol and/or carvacrol for use in treatment, prevention or management of cellular malfunction, genome damage, disease or condition associated with altered mitochondrial function or reduced mitochondrial density, in an individual in need thereof.

The present invention also provides a composition for use in inducing autophagy in an individual in need thereof. The composition comprises an effective amount of thymol and/or carvacrol, alone or in combination with high protein. The high amount of protein can be an amount of the protein that is at least about 25 energy % of the composition, and/or the high amount of protein can be an amount of the protein that provides a protein/energy ratio greater than 6 g/100 kcal of the composition.

In an embodiment, thymol and carvacrol are combined with an autophagy inducer selected from the group consisting of spermidine, urolithin (e.g., Urolithin A, B or D), rapamycin, Torin1, valproic acid, polyphenols (e.g., resveratrol), caffeine, metformin, 5′ AMP-activated protein kinase (AMPK) activators, L-type calcium channel inhibitors, ketones (e.g., beta-hydroxybutyrate, ketone salts, or ketone ester derivatives), and mixtures thereof.

In an embodiment, the autophagy is induced in skeletal muscle.

In an embodiment, the individual is an ageing individual.

In an embodiment, the individual has sarcopenia or frailty or is at risk of developing sarcopenia or frailty.

In an embodiment, the individual is critically ill.

In an embodiment, the individual has critical illness myopathy or is at risk of developing critical illness myopathy.

In an embodiment, the individual has a critical illness with acute kidney failure or is at risk of developing acute kidney failure.

In an embodiment the individual has a neurodegenerative disease or stroke.

In an embodiment the individual has a liver disease, e.g. NAFLD, NASH or a gastrointestinal condition such as intestinal inflammation, such as colitis ulcerosa, Crown's, mucositis and gut dysbiosis, for example.

In an embodiment, the individual has a chronic kidney disease with or without related loss of muscle mass or function.

In an embodiment, the individual has cachexia or muscle wasting secondary to a chronic disease such as cancer, chronic obstructive pulmonary disease (COPD), chronic heart failure (CHF), acute kidney disease or chronic kidney disease (CKD).

In an embodiment, at least a portion of the protein is selected from the group consisting of (i) protein from an animal source, (ii) protein from a plant source and (iii) a mixture thereof.

In an embodiment, at least a portion of the protein is selected from the group consisting of (i) milk protein, (ii) whey protein, (iii) caseinate, (iv) micellar casein, (v) pea protein, (vi) soy protein and (vii) mixtures thereof.

In an embodiment, the protein has a formulation selected from the group consisting of (i) at least 50 wt. % of the protein is casein, (ii) at least 50 wt. % of the protein is whey protein, (iii) at least 50 wt. % of the protein is pea protein and (iv) at least 50 wt. % of the protein is soy protein.

In an embodiment, at least a portion of the protein is selected from the group consisting of (i) free form amino acids, (ii) unhydrolyzed protein, (iii) partially hydrolyzed protein, (iv) extensively hydrolyzed protein, and (v) mixtures thereof. In a particular non-limiting example, at least a portion of the protein is collagen, i.e., unhydrolyzed and/or hydrolyzed collagen.

The protein can comprise one or more amino acids selected from the group consisting of histidine, isoleucine, leucine, lysine, methionine, phenylalanine, threonine, tryptophan, valine, arginine, cysteine, glutamine, glycine, proline, ornithine, serine, tyrosine, and mixtures thereof. The protein can comprise peptides having a length of 2 to 10 amino acids.

In an embodiment, the composition comprises branched chain amino acids in at least one form selected from the group consisting of (i) free form, (ii) bound to at least one additional amino acid, and (iii) mixtures thereof.

In an embodiment, at least a portion of the protein is 5 to 95% hydrolyzed.

In an embodiment, the protein has a formulation selected from the group consisting of (i) at least 50% of the protein has a molecular weight of 1-5 kDa, (ii) at least 50% of the protein has a molecular weight of 5-10 kDa and (iii) at least 50% of the protein has a molecular weight of 10-20 kDa.

In an embodiment, the composition comprises a carbohydrate source. The composition can have a high protein: carbohydrate ratio.

In an embodiment, the administering uses at least one route selected from the group of oral, enteral, parenteral and intravenous injection.

In another embodiment, the present disclosure provides a composition comprising a combination of thymol and/or carvacrol optionally with high protein, and the composition comprises an amount of the combination per serving that is effective to induce autophagy in an individual in need thereof. The composition can be selected from the group consisting of food compositions, dietary supplements, nutritional compositions, nutraceuticals, powdered nutritional products to be reconstituted in water or milk before consumption, food additives, medicaments, drinks, and combinations thereof.

In another embodiment, the present disclosure provides a method of making a therapeutic composition, the method comprising adding a combination of thymol and/or carvacrol alone or in combination with high protein to a base composition to form the therapeutic composition, the therapeutic composition comprising an amount of the combination per serving that is effective to induce autophagy in an individual in need thereof. The base composition can be formulated for administration by at least one route selected from the group of oral, enteral, parenteral and intravenous injection.

In another embodiment, the composition comprising thymol and/or carvacrol alone or in combination with high protein, concomitantly promotes protein synthesis and removal of damaged cellular materials to an individual in need thereof.

In another embodiment, the present disclosure provides a method of achieving at least one result selected from the group consisting of (i) an increased level of LC3-II protein expression or turnover, (ii) an increased level of the LC3-II/LC3-I protein ratio, (iii) a decreased level of p62 protein, (iv) a decreased level of a protein of the autophagosome, (v) an increased level of mRNA expression of an autophagy-related gene, (vi) an increased number and/or size and/or intensity of LC3 positive puncta, and (vii) degradation of LC3 and/or another autophagosome protein. The method comprises administering a therapeutically effective amount of a composition comprising a combination of thymol and/ or carvacrol, optionally with high protein to an individual in need thereof.

An advantage of one or more embodiments provided by the present disclosure is to improve the condition of individuals, critically ill animals, critically ill humans, ageing animals, or ageing humans.

Another advantage of one or more embodiments provided by the present disclosure is to prevent or treat excessive catabolism, e.g., in a critically ill patient, in an individual or an ageing individual.

Still another advantage of one or more embodiments provided by the present disclosure is to reduce or prevent the risk of morbidity or mortality due to excessive catabolism.

An additional advantage of the present disclosure is to reverse, treat or cure multiple organ dysfunction syndrome in a critically ill patient.

An additional advantage of one or more embodiments provided by the present disclosure is to protect an ageing individual from neurological diseases, such as mild cognitive impairment, Alzheimer disease, Parkinson's disease, Amyloid Lateral Sclerosis, Multiple Sclerosis, Huntington disease, dementia, and related neurological orphan diseases.

An additional advantage of one or more embodiments provided by the present disclosure is to protect an ageing individual from muscle dysfunction, for example sarcopenia, frailty, inclusion body myositis, myopathy/myolysis induced by drugs such as corticosteroids or statins, muscle wasting induced by immobilization or hospitalization.

An additional advantage of one or more embodiments provided by the present disclosure is to protect a patient suffering from a genetic disease, including but not restricted to muscular dystrophies such as Duchenne Muscular Dystrophy or Collagen VI muscular dystrophy, mitochondrial encephalomyopathies, mitochondrial myopathies, glycogen storage diseases, lysosmal storage diseases, Pompe disease.

Another advantage of one or more embodiments provided by the present disclosure is a composition that can be administered parenterally or enterally, for example as an aqueous liquid composition, to an individual or a critically ill patient to induce autophagy.

Yet another advantage of one or more embodiments provided by the present disclosure is to decrease a length of time that a critically ill patient spends on a ventilator or to accelerate the weaning time from a ventilator.

Another advantage of one or more embodiments provided by the present disclosure is to protect a critically ill patient subjected to parenteral nutrition, e.g., against multiple organ failure or muscle weakness caused by parenteral nutrient delivery, particularly unbalanced or relative nutrient overload.

An additional advantage of one or more embodiments provided by the present disclosure is to protect an ageing individual from muscle weakness.

Still another advantage of one or more embodiments provided by the present disclosure is to increase the survivability of a critically ill patient or an ageing individual.

An additional advantage of one or more embodiments provided by the present disclosure is to accelerate the regain of mobility, or shorten the time of immobility, after discharge from the intensive care unit.

Yet another advantage of one or more embodiments provided by the present disclosure is a beneficial effect even when a critically ill patient is already at a far-developed stage of a life threatening condition.

Additional features and advantages are described in, and will be apparent from, the following Detailed Description and the Figures.

### BRIEF DESCRIPTION OF DRAWINGS

FIG. 1 is a graph table showing that thymol induced autophagy in a dose dependent manner starting at the concentration of 125 uM in human Jurkat cells. Data are represented as mean ±SEM of 2 replicates, One-way ANOVA with post-hoc Dunnett's test. ***P<0.001.

FIG. 2 is a graph showing that thymol induced autophagy in a dose dependent manner starting at the concentration of 50 μM in zebrafish larvae. Data are represented as mean ±SEM of 18 replicates. Asterisks represent the significance levels calculated by one-way ANOVA with post-hoc Dunnett's test compared to untreated zebrafish ***P<0.001. Hash marks represent the significance levels calculated by one-way ANOVA with post-hoc Dunnett's test compared to NH4Cl treated zebrafish #P<0.05, ###P<0.001

FIG. 3 is a graph showing densitometric quantification of LC3-II/LC3-I protein amount of western blot performed on livers of mice treated in acute with thymol. Data are represented as mean ±SEM of 5 replicates, One-way ANOVA with post-hoc Dunnett's test. *P<0.05.

FIG. 4 is a graph showing reduction of liver steatosis in obese mice treated with thymol 20 mg/kg/day for 8 weeks. Data are represented as mean ±SEM of 12 replicates, one-tailed Student's t test for comparison has been used. *P<0.05, ***P<0.001.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment comprises more than 50 wt. % of the identified components, preferably at least 75 wt. % of the identified components, more preferably at least 85 wt. % of the identified components, most preferably at least 95 wt. % of the identified components, for example at least 99 wt. % of the identified components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage, e.g., an animal capable of autophagy. As used herein, the term “patient” is understood to include an animal, for example a mammal, and preferably a human that is receiving or intended to receive treatment, as treatment is herein defined. While the terms “individual” and “patient” are often used herein to refer to a human, the present disclosure is not so limited.

Accordingly, the terms “individual” and “patient” refer to any animal, mammal or human that can benefit from the methods and compositions disclosed herein. Indeed, non-human animals undergo prolonged critical illness that mimics the human condition. These critically ill animals undergo the same metabolic, immunological and endocrine disturbances and development of organ failure and muscle wasting as the human counterpart. Moreover, animals experience the effects of ageing as well.

The term “elderly” in the context of a human means an age from birth of at least 55 years, preferably above 63 years, more preferably above 65 years, and most preferably above 70 years. The term “older adult” or “ageing individual” in the context of a human means an age from birth of at least 45 years, preferably above 50 years, more preferably above 55 years, and includes elderly individuals.

For other animals, an “older adult” or “ageing individual” has exceeded 50% of the average lifespan for its particular species and/or breed within a species. An animal is considered “elderly” if it has surpassed 66% of the average expected lifespan, preferably if it has surpassed the 75% of the average expected lifespan, more preferably if it has surpassed 80% of the average expected lifespan. An ageing cat or dog has an age from birth of at least about 5 years. An elderly cat or dog has an age from birth of at least about 7 years.

“Sarcopenia” is defined as the age-associated loss of muscle mass and functionality (including muscle strength and gait speed). Sarcopenia can be characterized by one or more of low muscle mass, low muscle strength and low physical performance.

Sarcopenia can be diagnosed in a subject based on the definition of the AWGSOP (Asian Working Group for Sarcopenia in Older People), for example as described in Chen et al., 2014. Low muscle mass can generally be based on low appendicular lean mass normalized to height square (ALM index), particularly ALM index less than 7.00 kg/m2 for men and 5.40 kg/m2 for women. Low physical performance can generally be based on gait speed, particularly gait speed of <0.8 m/sec. Low muscle strength can generally be based on low hand grip strength, particularly hand grip strength less than 26 kg in men and less than 18 kg in women.

Additionally or alternatively, sarcopenia can be diagnosed in a subject based on the definition of the EWGSOP (European Working Group for Sarcopenia in Older People), for example as described in Crutz-Jentoft et al., 2010. Low muscle mass can generally be based on low appendicular lean mass normalized to height square (ALM index), particularly ALM index less than 7.23 kg/m2 for men and 5.67 kg/m2 for women. Low physical performance can generally be based on gait speed, particularly gait speed of <0.8 m/sec. Low muscle strength can generally be based on low hand grip strength, particularly hand grip strength less than 30 kg in men and less than 20 kg in women.

Additionally or alternatively, sarcopenia can be diagnosed in a subject based on the definition of the Foundation for the National Institutes of Health (FNIH), for example as described in Studenski et al., 2014. Low muscle mass can generally be based on low appendicular lean mass (ALM) normalized to body mass index (BMI; kg/m²), particularly ALM to BMI less than 0.789 for men and 0.512 for women. Low physical performance can generally be based on gait speed, particularly gait speed of <0.8 m/sec. Low muscle strength can generally be based on low hand grip strength, particularly hand grip strength less than 26 kg in men and less than 16 kg in women. Low muscle strength can also generally be based on low hand grip strength to body mass index, particularly hand grip strength to body mass index less than 1.00 in men and less than 0.56 in women.

As used herein, “frailty” is defined as a clinically recognizable state of increased vulnerability resulting from aging-associated decline in reserve and function across multiple physiologic systems such that the ability to cope with everyday or acute stressors is compromised. In the absence of an established quantitative standard, frailty has been operationally defined by Fried et al. as meeting three out of five phenotypic criteria indicating compromised energetics: (1) weakness (grip strength in the lowest 20% of population at baseline, adjusted for gender and body mass index), (2) poor endurance and energy (self-reported exhaustion associated with \dot{V}O₂ max), (3) slowness (lowest 20% of population at baseline, based on time to walk 15 feet, adjusting for gender and standing height), (4) low physical activity (weighted score of kilocalories expended per week at baseline, lowest quintile of physical activity identified for each gender; e.g., less than 383 kcal/week for males and less than 270 kcal/week for females), and/or unintentional weight loss (10 lbs. in past year). Fried L P, Tangen C M, Walston J, et al., “Frailty in older adults: evidence for a phenotype.” J. Gerontol. A. Biol. Sci. Med. Sci. 56(3):M146-M156 (2001). A pre-frail stage, in which one or two of these criteria are present, identifies a high risk of progressing to frailty.

“Cachexia” is a complex metabolic syndrome associated with underlying illness and characterized by loss of muscle with or without loss of fat mass. The prominent clinical feature of cachexia is weight loss in adults (corrected for fluid retention) or growth failure in children (excluding endocrine disorders).

Cachexia is often seen in patients with diseases such as cancer, chronic heart failure, renal failure, chronic obstructive pulmonary disease, AIDS, autoimmune disorders, chronic inflammatory disorders, cirrhosis of the liver, anorexia, chronic pancreatitis and/or metabolic acidosis and neurodegenerative disease.

There are certain types of cancer wherein cachexia is particularly prevalent, for example, pancreas, esophagus, stomach, bowel, lung and/or liver cancer.

The internationally recognized diagnostic criterion for cachexia is weight loss greater than 5% over a restricted time, for example 6 months, or weight loss greater than 2% in individuals already showing depletion according to current body weight and height (body-mass index [BMI] <20 kg/m²) or skeletal muscle mass (measured by DXA, MRI, CT or bioimpedance). Cachexia can develop progressively through various stages—precachexia to cachexia to refractory cachexia. Severity can be classified according to degree of depletion of energy stores and body protein (BMI) in combination with degree of ongoing weight loss.

In particular, cancer cachexia has been defined as weight loss >5% over past 6 months (in absence of simple starvation); or BMI<20 and any degree of weight loss >2%; or appendicular lean mass consistent with low muscle mass (males<7·26 kg/m²; females<5·45kg/m²) and any degree of weight loss >2% (Fearon et al. 2011). “Precachexia” may be defined as weight loss ≤5% together with anorexia and metabolic change. At present there are no robust biomarkers to identify those precachectic patients who are likely to progress further or the rate at which they will do so. Refractory cachexia is defined essentially on the basis of the patient's clinical characteristics and circumstances.

The terms “treatment” and “treating” include any effect that results in the improvement of the condition or disorder, for example lessening, reducing, modulating, or eliminating the condition or disorder. The term does not necessarily imply that a subject is treated until total recovery. Non-limiting examples of “treating” or “treatment of” a condition or disorder include: (1) inhibiting the condition or disorder, i.e., arresting the development of the condition or disorder or its clinical symptoms and (2) relieving the condition or disorder, i.e., causing the temporary or permanent regression of the condition or disorder or its clinical symptoms. A treatment can be patient-or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. The terms “condition” and “disorder” mean any disease, condition, symptom, or indication.

The relative terms “improved,” “increased,” “enhanced” and the like refer to the effects of the composition comprising a combination of autophagy inducer (e.g., spermidine) and high protein (disclosed herein) relative to a composition with less protein but otherwise identical.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

As used herein, “complete nutrition” contains sufficient types and levels of macronutrients (protein, fats and carbohydrates) and micronutrients to be sufficient to be a sole source of nutrition for the animal to which the composition is administered. Individuals can receive 100% of their nutritional requirements from such complete nutritional compositions.

As used herein, the term “critically ill patient” is an individual experiencing an acute life-threatening episode or diagnosed to be in imminent danger of such an episode. A critically ill patient is medically unstable and, when not treated, likely to die (e.g., >50% chance of death).

Non-limiting examples of critically ill patients include a patient who has sustained or is at risk of sustaining acutely life-threatening single or multiple organ system failure due to disease or injury, a patient who is being operated upon and where complications supervene, and a patient who has a vital organ operated upon within the last week or who has been subject to major surgery within the last week.

More specific non-limiting examples of a critically ill patient include a patient who has sustained or is at risk of sustaining acutely life-threatening single or multiple organ system failure due to disease or injury and a patient who is being operated upon and where complications supervene. Additional specific non-limiting examples of a critically ill patient include a patient in need of one or more of cardiac surgery, cerebral surgery, thoracic surgery, abdominal surgery, vascular surgery, or transplantation; and a patient suffering from one or more of a neurological disease, cerebral trauma, respiratory insufficiency, abdominal peritonitis, multiple trauma, a severe burn, or critical illness polyneuropathy.

The term “Intensive Care Unit” (ICU) refers to the part of a hospital where critically ill patients are treated. The term “Intensive Care Unit” also covers a nursing home; a clinic, for example, a private clinic; or the like if the treatment activities performed there are the same or similar as those of an ICU. An “ICU patient” is encompassed by the term “critically ill patient.”

The term “multiple organ dysfunction” refers to a condition resulting from infection, hypoperfusion, hypermetabolism or injury such as accident or surgery. The “multiple organ failure” of which critically ill patients die is considered a descriptive clinical syndrome defined by a dysfunction or failure of at least two vital organ systems. The vital organ systems that are uniformly and most specifically affected are the liver, the kidneys, the lungs, as well as the cardiovascular system, the nervous system and the hematological system. Non-limiting examples of multiple organ dysfunction include acute respiratory distress syndrome, heart failure, liver failure, renal failure, respiratory insufficiency, intensive care, shock, extensive burns, sepsis (e.g., systemic inflammatory response syndrome) and stroke.

The term “enterally administering” encompasses oral administration (including oral gavage administration), as well as rectal administration, although oral administration is preferred. The term “parenterally administering” refers to delivery of substances given by routes other than the digestive tract and covers administration routes such as intravenous, intra-arterial, intramuscular, intracerebroventricular, intraosseous, intradermal, intrathecal, and also intraperitoneal administration, intravesical infusion and intracavernosal injection.

Preferred parenteral administration is intravenous administration. A particular form of parenteral administration is delivery by intravenous administration of nutrition. Parenteral nutrition is “total parenteral nutrition” when no food is given by other routes. “Parenteral nutrition” is preferably a isotonic or hypertonic aqueous solution (or solid compositions to be dissolved, or liquid concentrates to be diluted to obtain an isotonic or hypertonic solution) comprising a saccharide such as glucose and further comprising one or more of lipids, amino acids, and vitamins.

### Embodiments

Accordingly, in a general embodiment, the present disclosure provides a composition comprising thymol and/or carvacrol for use in treatment, prevention or management of cellular malfunction, genome damage, disease or condition associated with altered mitochondrial function or reduced mitochondrial density, in an individual in need thereof. It also provides a composition for induction of autophagy.

In an aspect of the present invention, the composition increases antioxidant capacity, reduces oxidative stress, maintains immune function and/or maintains cognitive function in a healthy older adult.

In another aspect, the mitochondria-related disease or condition is selected from the group consisting of stress, obesity, reduced metabolic rate, metabolic syndrome, diabetes mellitus, complications from diabetes, hyperlipidemia, elevated free fatty acids, liver disease, NAFLD, NASH, neurodegenerative disease, stroke, cognitive disorder, stress-induced or stress-related cognitive dysfunction, mood disorder, anxiety disorder, age-related neuronal death or dysfunction, musculoskeletal disorder, frailty, pre-frailty, chronic kidney disease, gastrointestinal disorder, trauma, infection, cancer, macular degeneration, and combinations thereof.

Another aspect of the present invention is a method of inducing autophagy in an individual in need thereof. The method comprises administering a composition comprising thymol and/or carvacrol, optionally with high protein (e.g., about 25% of the total energy of the composition), and the composition is administered to provide an amount of the combination that is effective to induce autophagy, for example in muscle. The composition can be administered parenterally, enterally, or intravenously.

In a preferred embodiment, the composition further contains an autophagy inducer selected from the group consisting of spermidine, urolithin (e.g., Urolithin A, B or D), rapamycin, Torin1, valproic acid, polyphenols (e.g., resveratrol), caffeine, metformin, 5′ AMP-activated protein kinase (AMPK) activators, L-type calcium channel inhibitors, and mixtures thereof. Non-limiting examples of suitable autophagy inducers are spermidine, palmitic acid, 5-aminoimidazole-4-carboxamide riboside (AICAR), verapamil, nifedipine, diltiazem, piperazine phenothiazine derivatives (e.g., trifluoperazine), ketones (e.g., beta-hydroxybutyrate, ketone salts, or ketone ester derivatives) and mixtures thereof. Non-limiting examples of suitable forms of spermidine include spermidine trihydrochloride, spermidine phosphate hexahydrate, spermidine phosphate hexahydrate, and L-arginyl-3,4-spermidine.

In an embodiment, the composition has a protein/energy ratio greater than 6 g protein/ 100 kcal, preferably greater than 9 g protein/100 kcal. In an embodiment, the protein is at least 24 energy % of the composition and more preferably at least 36 energy % of the composition.

As non-limiting examples, the composition can be administered in a daily dose that provides an amount of protein greater than 1.0 g protein/kg body weight/day, preferably greater than 1.2 g protein/kg body weight/day; for example up to 2.5 g protein/kg body weight/day (e.g., 1.0-2.5 g protein/kg body weight/day; 1.2-2.5 g protein/kg body weight/day; or 1.5-2.5 g protein/kg body weight/day), preferably up to 2.0 g protein/kg body weight/day (e.g., 1.0-2.0 g protein/kg body weight/day; 1.2-2.0 g protein/kg body weight/day; or 1.5-2.0 g protein/kg body weight/day), and more preferably up to 1.5 g protein/kg body weight/day (e.g., 1.0-1.5 g protein/kg body weight/day or 1.2-1.5 g protein/kg body weight/day). The daily dose of the protein can be provided by one or more servings of the composition per day.

If the composition is in liquid form, non-limiting examples of suitable high protein concentrations include 6-20 g protein/ 100 ml, e.g., 6-11 g protein/100 ml; 7-14 g protein/ 100 ml; 7-12 g protein/ 100 ml; 8-11 g protein/ 100 ml, 8-20 g protein/100 ml; 9-20 g protein/ 100 ml; and 11-20 g protein/100 ml.

The composition can comprise a pharmacologically effective amount of thymol and/or carvacrol in a pharmaceutically suitable carrier. In aqueous liquid compositions, concentration preferably ranges from about 0.05 wt. % to about 4 wt. %, or from about 0.5 wt. % to about 2 wt. % or from about 1.0 wt. % to about 1.5 wt. % of the aqueous liquid composition.

In particular embodiments, the method is a treatment that augments the plasma thymol and/or carvacrol level in an individual, for example to a level in the range of 50 to 6000 nmol/L plasma, preferably 100 to 6000 nmol/L plasma. The method can comprise administering daily thymol and/or carvacrol in the weight range of 0.05 mg-1 g per kg body weight, preferably 1 mg-200 mg per kg body weight, more preferably 5 mg-150 mg per kg body weight, even more preferably 10 mg-120 mg per kg body weight, or most preferably 40 mg-80 mg per kg body weight.

Typically between 50 μg to 10 g of thymol and/or carvacrol, per daily serving in one or more portions is administered to an individual. Thymol (10-64%) is one of the major constituent of essential oils of thyme (Thymus vulgaris L., Lamiaceae). Carvacrol is present in the essential oil of Origanum vulgare (oregano), oil of thyme, oil obtained from pepperwort, and wild bergamot. The essential oil of thyme subspecies contains between 5% and 75% of carvacrol, while Satureja (savory) subspecies have a content between 1% and 45%. Origanum majorana (marjoram) and Dittany of Crete are rich in carvacrol, 50% and 60-80% respectively. Therefore, some embodiments of the composition comprise such plant and/or enriched plant extracts, essential oils or fractions that provide at least a portion of thymol and/carvacrol in the composition, in particular from thyme and oregano.

The composition can induce autophagy in muscle, for example a skeletal muscle. Non-limiting examples of such muscle include one or more of the following: vastus lateralis, gastrocnemius, tibialis, soleus, extensor, digitorum longus (EDL), biceps femoris, semitendinosus, semimembranosus, gluteus maximus, extra-ocular muscles, face muscles or diaphragm.

The individual in need of induced autophagy can be an individual having mitochondria-related disease or condition, which is selected from the group consisting of stress, obesity, reduced metabolic rate, metabolic syndrome, diabetes mellitus, complications from diabetes, hyperlipidemia, elevated free fatty acids, liver disease, NAFLD, NASH, neurodegenerative disease, stroke, cognitive disorder, stress-induced or stress-related cognitive dysfunction, mood disorder, anxiety disorder, age-related neuronal death or dysfunction, musculoskeletal disorder, frailty, pre-frailty, chronic kidney disease, gastrointestinal disorder (such as intestinal inflammation, such as colitis ulcerosa, Crown's, mucositis and gut dysbiosis), trauma, infection, cancer, macular degeneration, and combinations thereof.

The individual in need of induced autophagy can be an ageing individual, such as an ageing animal or an ageing human. In some embodiments, the individual in need of induced autophagy is an elderly animal or an elderly human.

The individual in need of induced autophagy can be a critically ill patient. In various embodiments, the method can treat or prevent multiple organ dysfunction in the critically ill patient, e.g., if the patient has failed or disturbed homeostasis from receiving parenteral nutrition; can protect the critically ill patient against multiple organ dysfunction; can treat or prevent development of lactic acidosis, for example lactic acidosis induced by parenteral nutrition; can treat or prevent muscle weakening in the critically ill patient; can decrease or prevent morbidity or mortality nutrition aggravated by parenteral nutrition; and/or can prevent body system collapse.

In some embodiments, the critically ill patient has at least one life threatening condition selected from the group consisting of lactic acidosis, muscle weakening, hyperglycemia, multiple organ failure, failed homeostasis, and disturbed homeostasis. In an embodiment, the critically ill patient has a non-infectious disorder. In an embodiment, the critically ill patient has multiple organ dysfunction that is not caused or associated with sepsis. Multiple organ dysfunction and muscle weakness are common in the critical care setting and can be caused or aggravated by unbalanced parenteral nutrient delivery or a parenterally delivered relative or absolute nutrient overload.

In some embodiments, the individual or critically ill patient has at least one disorder selected from the group consisting of severe trauma, multiple trauma, stroke, neurodegenerative disease, high risk surgery, extensive surgery, cerebral trauma, cerebral bleeding, respiratory insufficiency, abdominal peritonitis, acute kidney injury, acute liver injury, NAFLD, NASH, gastrointestinal disorders (such as intestinal inflammation, such as colitis ulcerosa, Crown's, mucositis and gut dysbiosis), severe burns, critical illness polyneuropathy, critical illness myopathy, and ICU-acquired muscle weakness.

In some embodiments, the individual or critically ill patient is receiving enteral or parenteral nutrition. In some embodiments, the composition treats or prevents mitochondrial dysfunction, for example mitochondrial dysfunction induced by inadequate or unbalanced parenteral nutrition to a critically ill patient.

In another aspect of the present disclosure, a method achieves at least one result selected from the group consisting of: an increased level of LC3-II protein expression or turnover (e.g., as can be measured by western blot, mass-spectrometry, ELISA, aptamer-or nanobody-based proteomics); an increased level of the LC3-II/LC3-I protein ratio (e.g., as can be measured by any of the methods above); a decreased level of p62 protein (e.g., as can be measured by the aforementioned methods); a decreased level of a protein of the autophagosome, for example but not limited to Atg5, Beclin-1, Atg7, Atg12; an increased level of mRNA expression of autophagy related genes, for example but not limited to MAP1LC3, GABARAP, Atg5, Beclin-1, Atg7, Atg12; and increased number and/or size and/or intensity of LC3 positive puncta (as can be assessed by immunofluorescence or by tagging LC3 to a fluorescent reporter protein like GFP or by flow cytometry); degradation of LC3 and/or another autophagosome protein (as can be measured by assessing its lysosomal degradation assessed by microscopy or flow cytometry, for example by fusing the protein to a pH sensitive fluorescent reporter that will change color when reaching the lysosome; or can be measured by comparing the fluorescent intensity of the WT protein to a mutated protein which cannot be inserted in autophagosomes, for example, the LC3ΔG mutant which cannot be lipidated and inserted in the autophagosome). The method comprises administering a therapeutically effective amount of a composition comprising a combination of an autophagy inducer and high protein to an individual in need thereof.

The term “protein” as used herein includes free form amino acids, molecules between 2 and 20 amino acids (referenced herein as “peptides”), and also includes longer chains of amino acids as well. Small peptides, i.e., chains of 2 to 10 amino acids, are suitable for the composition alone or in combination with other proteins. The “free form” of an amino acid is the monomeric form of the amino acid. Suitable amino acids include both natural and non-natural amino acids. The composition can comprise a mixture of one or more types of protein, for example one or more (i) peptides, (ii) longer chains of amino acids, or (iii) free form amino acids; and the mixture is preferably formulated to achieve a desired amino acid profile/content.

At least a portion of the protein can be from animal or plant origin, for example dairy protein such as one or more of milk protein, e.g., milk protein concentrate or milk protein isolate; caseinates or casein, e.g., micellar casein concentrate or micellar casein isolate; or whey protein, e.g., whey protein concentrate or whey protein isolate. Additionally or alternatively, at least a portion of the protein can be plant protein such as one or more of soy protein or pea protein.

Mixtures of these proteins are also suitable, for example mixtures in which casein is the majority of the protein but not the entirety, mixtures in which whey protein is the majority of the protein but not the entirety, mixtures in which pea protein is the majority of the protein but not the entirety, and mixtures in which soy protein is the majority of the protein but not the entirety. In an embodiment, at least 10 wt. % of the protein is whey protein, preferably at least 20 wt. %, and more preferably at least 30 wt. %. In an embodiment, at least 10 wt. % of the protein is casein, preferably at least 20 wt. %, and more preferably at least 30 wt. %. In an embodiment, at least 10 wt. % of the protein is plant protein, preferably at least 20 wt. %, more preferably at least 30 wt. %.

Whey protein may be any whey protein, for example selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof.

Casein may be obtained from any mammal but is preferably obtained from cow milk and preferably as micellar casein.

The protein may be unhydrolyzed, partially hydrolyzed (i.e., peptides of molecular weight 3 kDa to 10 kDa with an average molecular weight less than 5 kDa) or extensively hydrolyzed (i.e., peptides of which 90% have a molecular weight less than 3 kDa), for example in a range of 5% to 95% hydrolyzed. In some embodiments, the peptide profile of hydrolyzed protein can be within a range of distinct molecular weights. For example, the majority of peptides (>50 molar percent or >50 wt. %) can have a molecular weight within 1-5 kDa, or 5-10 kDa, or 10-20 kDa.

The protein can comprise essential amino acids and/or conditionally essential amino acids, e.g., such amino acids that may be insufficiently delivered in a caloric restriction regimen. For example, the protein can comprise one or more essential amino acids selected from the group consisting of histidine, isoleucine, leucine, lysine, methionine, phenylalanine, threonine, tryptophan, and valine; and each of these amino acids (if present) may be administered in the composition in a daily dose from about 0.0476 to about 47.6 mg amino acid/kg bw. Notably, lower intake of methionine leads to lower levels of protein translation and ultimately muscle synthesis. The protein can comprise one or more conditionally essential amino acids (e.g., amino acids conditionally essential in illness or stress) selected from the group consisting of arginine, cysteine, glutamine, glycine, proline, ornithine, serine and tyrosine; and each of these amino acids (if present) may be administered in the composition in a daily dose from about 0.0476 to about 47.6 mg amino acid/kg bw.

The composition can comprise one or more branched chain amino acids (BCAAs). For example, the composition can comprise leucine, isoleucine and/or valine, in free form and/or bound as peptides and/or proteins such as dairy, animal or plant proteins. A daily dose of the branched chain amino acids can include one or more of 0.35-142.85 mg/kg bw Leucine, preferably 0.175-71.425 mg/kg bw Leucine; 0.175-71.425 mg/kg bw Isoleucine; and 0.175-71.425 mg/kg bw Valine. The daily dose of the one or more branched chain amino acids can be provided by one or more servings of the composition per day.

Whey protein is rich in BCAAs. Therefore, some embodiments of the composition comprise whey protein that provides at least a portion of the BCAAs in the composition.

In an embodiment, the composition includes a source of carbohydrates. Any suitable carbohydrate may be used in the composition including, but not limited to, starch (e.g., modified starch, amylose starch, tapioca starch, corn starch), sucrose, lactose, glucose, fructose, corn syrup solids, maltodextrin, xylitol, sorbitol or combinations thereof.

The source of carbohydrates is preferably not greater than 50 energy % of the composition, more preferably not greater than 36 energy % of the composition, and most preferably not greater than 30 energy % of the composition. The composition can have a high protein: carbohydrate energy ratio, for example greater than 0.66, preferably greater than 0.9 and more preferably greater than 1.2.

In an embodiment, the composition includes a source of fat. The source of fat may include any suitable fat or fat mixture. Non-limiting examples of suitable fat sources include vegetable fat, such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like, animal fats such as milk fat; or combinations thereof.

The composition comprising a combination of thymol and/or carvacrol, optionally combined with high protein can be administered to an individual such as a human, e.g., an ageing individual or a critically ill individual, in a therapeutically effective dose. The therapeutically effective dose can be determined by the person skilled in the art and will depend on a number of factors known to those of skill in the art, such as the severity of the condition and the weight and general state of the individual.

The composition is preferably administered to the individual at least two days per week, more preferably at least three days per week, most preferably all seven days of the week; for at least one week, at least one month, at least two months, at least three months, at least six months, or even longer. In some embodiments, the composition is administered to the individual consecutively for a number of days, for example at least until a therapeutic effect is achieved. In an embodiment, the composition can be administered to the individual daily for at least 30, 60 or 90 consecutive days.

The above examples of administration do not require continuous daily administration with no interruptions. Instead, there may be some short breaks in the administration, such as a break of two to four days during the period of administration. The ideal duration of the administration of the composition can be determined by those of skill in the art.

In a preferred embodiment, the composition is administered to the individual orally or enterally (e.g. tube feeding). For example, the composition can be administered to the individual as a beverage, a capsule, a tablet, a powder or a suspension.

The composition can be any kind of composition that is suitable for human and/or animal consumption. For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In some instances where oral or enteral administration is not possible or not advised, the composition may also be administered parenterally.

In some embodiments, the composition is administered to the individual in a single dosage form, i.e. all compounds are present in one product to be given to an individual in combination with a meal. In other embodiments, the composition is co-administered in separate dosage forms, for example at least one component separately from one or more of the other components of the composition.

### EXAMPLE

### Example 1: In Vitro Experiment

### Material and Methods

The human lymphocytic T cell line Jurkat (clone E6.1, ATCC TIB-152) has been used to measure autophagic flux in vitro. Cells were grown in RPMI medium with standard conditions (5% CO2, 37° C.) in a humidified atmosphere. For the experiment, cells were washed, counted and incubated at 1*105/well in duplicate in a 96-flat bottom well plate. Experimental conditions included negative controls (0.5% DMSO), rapamycin treatment as positive control (1 uM) and Earle'sBalanced Salt Solution (EBSS) as a starvation medium. Cells received Thymol (in RPMI) at different concentrations ranging from 1.95 μM to 250 μM. In parallel, the same experimental conditions were prepared for the incubation with solution A, from the Flow Cellect Autophagy Kit from Merck, now Guava Autophagy LC3 antibody-based kit. The treatment with compound A blocks the lysosomal degradation of LC3 vesicles and is used to measure autophagic flux. Cells were incubated with thymol at 37° for 1.30 h. Solution A was added for additional 30 minutes. Cells were then transferred in a 96 V bottom plate for the LC3 antibody detection using the Guava Autophagy LC3 antibody-based kit, according the manufacturer's instructions. Briefly, cells were permeabilized to remove the cytoplasmic form of LC3 (LC3-I) and incubated with a monoclonal anti LC3 antibody conjugated to the fluorophore Fluoresceine Isothiocyanate (FITC). Samples were then acquired using a Becton Dickinson LSORP Fortessa flow cytometry analyzer. Offline analysis was performed with FCS Express Software and results were expressed as the median fluorescence intensity in the FITC channel (related to the amount of LC3-II present in the cells.)

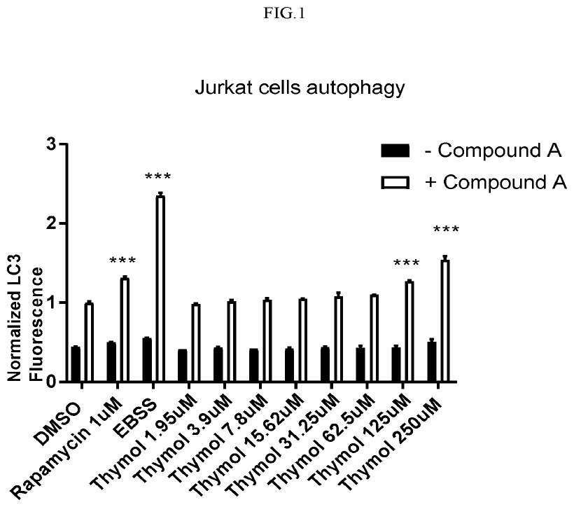

Results are provided in FIG. 1 which shows that thymol induced autophagy in a dose dependent manner starting at the concentration of 125 uM in the human Jurkat cells.

### Example 2: Experiments on Autophagy Reporter Zebrafish Line

### Material and Methods

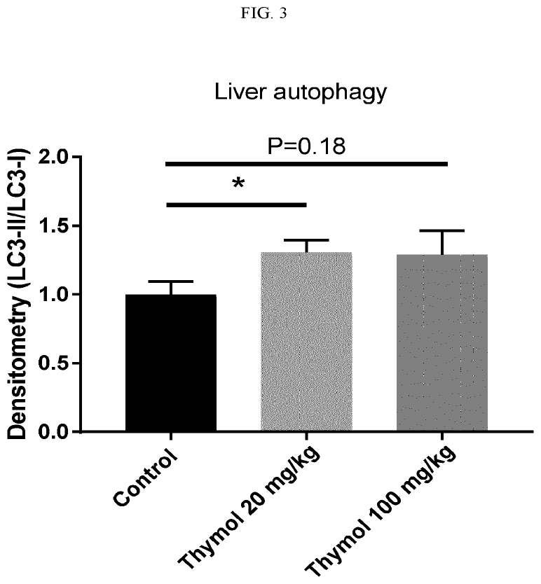

An autophagy reporter zebrafish line has been generated by stable expression of the LC3 protein fused to ZsGreen under the control of a skeletal muscle specific promoter. Larvae from outcrossed transgenic zebrafish were raised at 28° C. under standard laboratory conditions and have been treated at 48 h post-fertilization in 96 well plates with thymol at different concentrations as indicated in FIG. 3. After 16 hours of treatment larvae were anesthetized with 0.016% tricaine and imaged with ImageXpress confocal system at 20× magnification (Molecular Devices). Z stack images were captured for each larva and maximal projection images were produced. Ammonium chloride was added for additional 4 hours to block lysosomal degradation. Images were acquired again as before. In order to quantify autophagic flux, number of LC3 punctae have been calculated in presence and in absence of ammonium chloride with MetaXpress software (Molecular Devices) and normalized by zebrafish area.

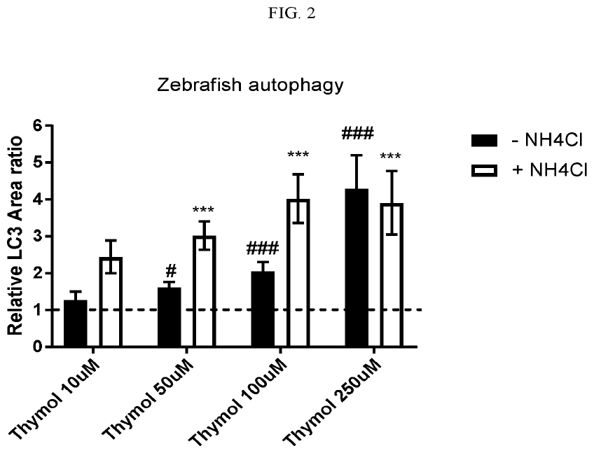

Results are reported in FIG. 2. This graph shows that thymol induced autophagy in a dose dependent manner starting at the concentration of 50 uM in the zebrafish larvae.

### Example 3: In-Vivo Experiments

### Material and Methods

### Acute Treatment

10-15 weeks-old C57bl6/J received two treatments with thymol at the concentrations of 20 mg/kg/body weight and 100 mg/kg/body weight on two consecutive days before tissues harvesting. Mice were sacrificed by isofluoran inhalation followed by exsanguination. Livers were collected and frozen in liquid nitrogen.

### Chronic Treatment on a Model of Obesity

Mice were fed with high fat diet (Research Diets D 12492: 60% fat, 20% protein, 20% carbohydrates) for 8 weeks. Mice were sacrificed by isofluoran inhalation followed by exsanguination. Livers were collected, embedded in OCT and frozen in isopentane. In order to visualize lipid droplets, liver sections of 10 μm were cut and stained by Oil Red O. Lipids size was calculated using imageJ software

### Western Blots

Total protein lysates were extracted from 30-50 mg of tissues homogenized in 20 ml/g of RIPA buffer (150 mM sodium chloride, 50 mM Tris pH: 8, 1% Triton X-100, 0.5% deoxycolate, 0.1% SDS, protease inhibitors cocktail) with a tissue dissociator (gentleMACS Miltenyi Biotec). Protein concentrations were determined by BCA assay and samples were prepared adding 4×LDS sample buffer (Invitrogen). 20 μg of proteins were separated by SDS-PAGE in 4-12% gradient gels and transferred to PVDF membranes using dry iBLOT system (Invitrogen). Membranes were incubated with LC3 (Novus Biologicals 2220) and GAPDH (Cell Signaling 2118) antibodies and detected with ECL substrates (Pierce). Protein quantification was performed by densitometric analysis of images using ImageJ software.

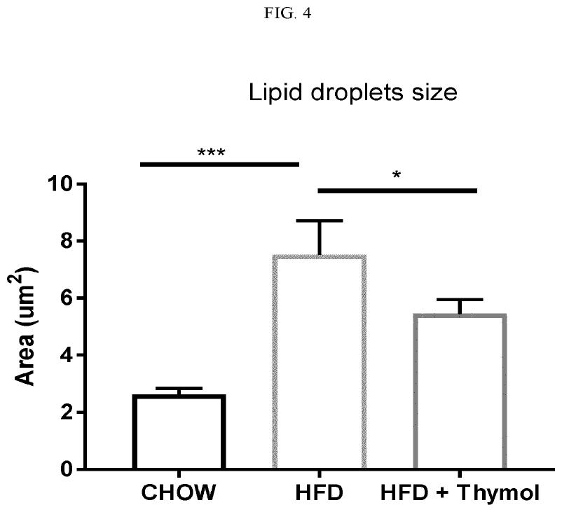

Results are presented respectively in FIGS. 3 and 4. FIG. 3, shows densitometric quantification of LC3-II/LC3-I protein amount of western blot performed on livers of mice treated in acute with thymol and FIG. 4 shows reduction of liver steatosis in obese mice treated with thymol 20 mg/kg/day for 8 weeks.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for treatment, prevention or management of cellular malfunction, genome damage, or a disease or condition associated with altered mitochondrial function or reduced mitochondrial density, the method comprising administering a composition comprising at least one component selected from the group consisting of thymol and carvacrol to an individual in need thereof.

2. The method according to claim 1, for increasing antioxidant capacity, reducing oxidative stress maintaining immune function and/or maintaining cognitive function in a healthy older adult.

3. The method according to claim 1, wherein the disease or condition associated with altered mitochondrial function or reduced mitochondrial density is selected from the group consisting of stress, obesity, reduced metabolic rate, metabolic syndrome, diabetes mellitus, complications from diabetes, hyperlipidemia, elevated free fatty acids, liver disease, NAFLD, NASH, neurodegenerative disease, stroke, cognitive disorder, stress-induced or stress-related cognitive dysfunction, mood disorder, anxiety disorder, age-related neuronal death or dysfunction, musculoskeletal disorder, frailty, pre-frailty, chronic kidney disease, gastrointestinal disorders, trauma, infection, cancer, macular degeneration, and combinations thereof.

4. The method of claim 1, wherein composition further comprises high protein.

5. The method according to claim 4, wherein at least a portion of the protein is selected from the group consisting of (i) milk protein, (ii) whey protein, (iii) caseinate, (iv) micellar casein, (v) pea protein, (vi) soy protein and (vii) mixtures thereof.

6. The method according to claim 1, wherein the composition further comprises a carbohydrate source.

7. The method according to claim 1, wherein the composition further comprises an autophagy inducer is selected from the group consisting of spermidine, urolithin, rapamycin, Torin1, valproic acid, polyphenols, caffeine, metformin, 5′AMP-activated protein kinase (AMPK) activators, L-type calcium channel inhibitors, ketones, and mixtures thereof.

8. The method according to claim 1, wherein the individual is an ageing individual.

9. The method according to claim 1, wherein the individual has sarcopenia or frailty or is at risk of developing sarcopenia or frailty.

10. The method according to claim 1, wherein the individual is critically ill.

11. A method comprising administering a composition comprising a combination of an autophagy inducer and high protein, the composition is administered to provide an amount of the combination that concomitantly promotes protein synthesis and removal of damaged cellular materials to an individual in need thereof.

12. A method of achieving at least one result selected from the group consisting of (i) an increased level of LC3-II protein expression or turnover, (ii) an increased level of the LC3-II/LC3-I protein ratio, (iii) a decreased level of p62 protein, (iv) a decreased level of a protein of the autophagosome, (v) an increased level of mRNA expression of an autophagy-related gene, (vi) an increased number and/or size and/or intensity of LC3 positive puncta, and (vii) degradation of LC3 and/or another autophagosome protein, the method comprising administering a therapeutically effective amount of a composition comprising a combination of an autophagy inducer and high protein to an individual in need thereof.
